(19) United States
(12) Patent Application Publication
(10) Pub. No.: US 2020/0043575 A1
(43) Pub. Date: Feb. 6, 2020

(54) Electronic system with a data engine for processing retrieved data in displaying graphical data for a graphical user interface on an external input/output device

(71) Applicant: Carnegie Mellon University, Pittsburgh, PA (US)

(72) Inventors: Armaghan W. Naik, Pittsburgh, PA (US); Joshua D. Kangas, Pittsburgh, PA (US); Christopher J. Langmead, Monroeville, PA (US); Robert F. Murphy, Wexford, PA (US)

(21) Appl. No.: 16/296,088

(22) Filed: Mar. 7, 2019

Related U.S. Application Data
(63) Continuation of application No. 13/985,247, filed on Oct. 28, 2013, now abandoned.
(60) Provisional application No. 61/463,593, filed on Feb. 18, 2011.
(60) Provisional application No. 61/463,589, filed on Feb. 18, 2011.
(60) Provisional application No. 61/463,206, filed on Feb. 14, 2011.

(51) Int. Cl. G16C 20/70; G16C 20/30

(52) U.S. Cl. CPC G16C 20/70; G01N 33/48; G16C 20/30

(57) ABSTRACT

An electronic computing system configured to process a plurality of data elements by performing operations including obtaining information indicative of features of data elements; generating training data for a machine learning engine with the features of the data elements; initializing the machine learning engine with the training data; processing the data elements with the machine learning engine; iteratively updating a model of the data elements based on the processing; and displaying a visual representation of the data elements in accordance with the updated model.

### GOVERNMENT RIGHTS

The techniques disclosed herein are made with government support under the National Institutes of Health, grant number 3R01 GM075205-03S2. The government may have certain rights in the techniques disclosed herein.

### BACKGROUND

Drug development is a lengthy process that begins with the identification of proteins involved in a disease and ends after testing in clinical trials. For a protein, drugs are identified that either increase or decrease an activity of a protein that is linked to a disease.

In an example, high throughput screening (HTS) is a common way to test the effects of many drugs on a protein. In HTS, an assay is used to detect effects of a drug on a protein. Generally, an assay includes a material that is used in determining the properties of another material.

### SUMMARY

In one aspect of the present disclosure, a method performed by one or more processing devices includes obtaining information indicative of experiments associated with combinations of targets and compounds; initializing the information with a result of at least one of the experiments; generating, based on initializing, a model to predict effects of the compounds on the targets; generating, based on the model and the experiments obtained, predictions for experiments to be executed; selecting, based on the predictions, one or more experiments from the experiments to be executed; executing the one or more experiments; and updating the model with one or more results of execution of the one or more experiments.

Implementations of the disclosure can include one or more of the following features. In some implementations, a prediction includes a value indicative of a whether a compound is predicted to have an effect on a target. In other implementations, the effect includes an active effect or an inactive effect. In yet other implementations, selecting includes: selecting, from the experiments to be executed, an experiment associated with a prediction of an increased effect, relative to other predictions of other effects of other of the experiments to be executed.

In some implementations, the method includes repeating the actions of generating the predictions, selecting, executing and updating, until detection of a pre-defined condition. In other implementations, the method includes retrieving information indicative of the targets and the compounds; wherein obtaining includes: generating, from the information obtained, an experimental space, wherein the experimental space comprises a visual representation of the information indicative of the experiments associated with the combinations of the targets and the compounds; and wherein updating includes updating the experimental space.

In some implementations, the method includes retrieving information indicative of features of one or more of the compounds and the targets; wherein generating the model includes: generating the model based on the features. In other implementations, a feature includes at least one of a molecular weight feature, a theoretical isoelectric point feature, an amino acid composition feature, an atomic composition feature, an extinction coefficient feature, an instability index feature, an aliphatic index feature, and a grand average of hydropathicity feature.

In some implementations, the model includes: generating the model independent of features of the compounds and the targets. In other implementations, a compound includes one or more of a drug, a combination of drugs, a nucleic acid, and a polymer; and a target includes one or more of a protein, an enzyme, and a nucleic acid.

In still another aspect of the disclosure, method performed by one or more processing devices includes obtaining information indicative of experiments associated with combinations of targets and compounds; initializing the information with a result of at least one of the experiments; generating, based on initializing, a model to predict effects of the compounds on the targets; selecting, based on features of one or more of the targets and the compounds and from the experiments obtained, one or more experiments for execution; executing the one or more experiments selected; and updating the model with one or more results of execution of the one or more experiments.

In still another aspect of the disclosure, one or more machine-readable media are configured to store instructions that are executable by one or more processing devices to perform one or more of the foregoing features.

In yet another aspect of the disclosure, an electronic system includes one or more processing devices; and one or more machine-readable media configured to store instructions that are executable by the one or more processing devices to perform one or more of the foregoing features.

All or part of the foregoing can be implemented as a computer program product including instructions that are stored on one or more non-transitory machine-readable storage media, and that are executable on one or more processing devices. All or part of the foregoing can be implemented as an apparatus, method, or electronic system that can include one or more processing devices and memory to store executable instructions to implement the stated functions.

The details of one or more implementations are set forth in the accompanying drawings and the description below. Other features, objects, and advantages will be apparent from the description and drawings, and from the claims.

### BRIEF DESCRIPTION OF DRAWINGS

FIG. 1 is a diagram of an example of a network environment for generating predictions of effects of compounds on targets.

FIG. 2 is a block diagram showing examples of components of a network environment for generating predictions of effects of compounds on targets.

FIG. 3 is a flowchart showing an example process for generating predictions of effects of compounds on targets.

FIG. 4 shows an example of a computer device and a mobile computer device that can be used to implement the techniques described herein.

Like reference symbols and designations in the various drawings indicate like elements.

### DETAILED DESCRIPTION

A system consistent with this disclosure measures and/or generates predictions of effects of compounds on targets. Generally, a target includes an item for which an effect can be measured. Types of targets includes proteins, enzymes, nucleic acids, and so forth. Generally, a compound includes a material. Types of compounds include drugs, combinations of drugs (e.g., drug cocktails) chemicals, polymers, nucleic acids, and so forth.

In an example, the system includes thousands of targets and millions of compounds. Using an active learning technique, the system is configured to generate measurements or predictions of the effect of all compounds on all targets.

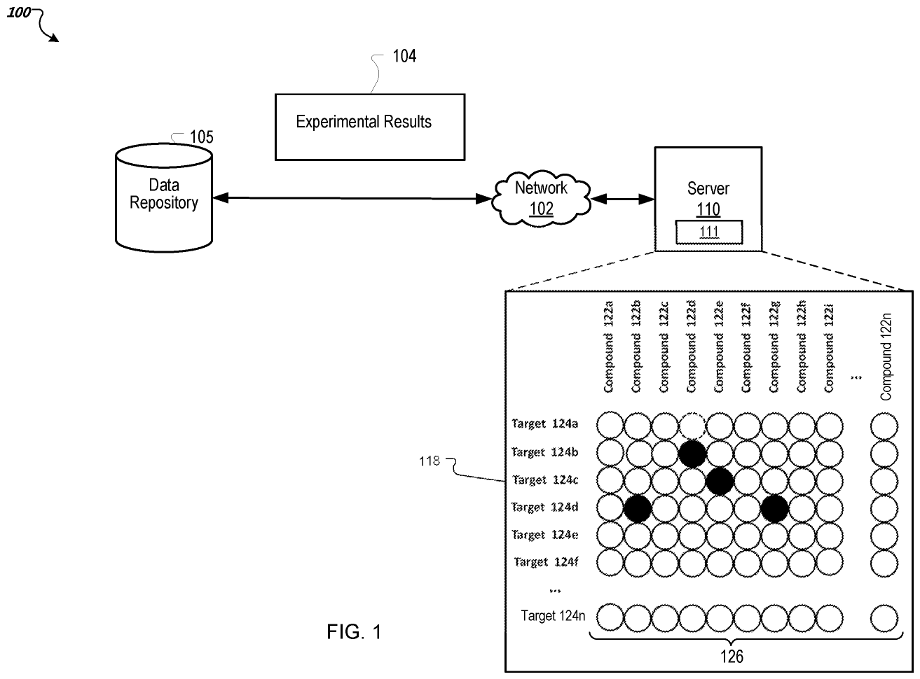

FIG. 1 is a diagram of an example of a network environment 100 for generating predictions of effects of compounds on targets. Network environment 100 includes network 102, data repository 105, and server 110.

Data repository 105 can communicate with server 110 over network 102. Network environment 100 may include many thousands of data repositories and servers, which are not shown. Server 110 may include various data engines, including, e.g., data engine 111. Although data engine 111 is shown as a single component in FIG. 1, data engine 111 can exist in one or more components, which can be distributed and coupled by network 102.

In the example of FIG. 1, data engine 111 retrieves, from data repository 105, information indicative of targets 124a . . . 124n and compounds 122a . . . 122n. In this example, data engine 111 is configured to execute experiments to predict effects of one or more of compounds 122a . . . 122n on one or more of targets 124a . . . 124n. Using targets 124a . . . 124n and compounds 122a . . . 122n, data engine 111 generates experimental space 118. Generally, experimental space 118 includes a visual representation of a set of experiments 126 ranging over targets 124a . . . 124n and compounds 122a . . . 122n. In this example, experiments 126 are visually represented as white circles, with black boundary lines.

In an example, experiments 126 include executed experiments and unexecuted experiments. Generally, an executed experiment includes an experiment that has been performed by data engine 111. An unexecuted experiment includes an experiment that has not yet been performed by data engine 111.

As experiments 126 are performed, data engine 111 may associate an experiment with an observation. Generally, an observation includes information indicative of an effect of a compound on a target. For example, an observation may include information indicative of whether a compound increases or decreases activity in a target.

Based on an observation from an experiment, data engine 111 may annotate an experiment. As described in further detail below, an experiment may be annotated changing the color of the circle to black and/or by changing the boundary line to be a dashed line.

In an example, data engine 111 retrieves, from data repository 105, experimental results 104. In this example, experimental results 104 include information indicative of results of experiments that have been previously performed by an entity. For example, experimental results 104 may include PubChem assay data, including, e.g., information about compounds tested with an assay for a target.

In this example, experimental results 104 include information indicative of results of compound 122b on target 124d, results of compound 122d on targets 124a-124b, results of compound 122e on target 124c, and results of compound 122g on target 124d. A result includes an active result, an inactive result, and so forth. Generally, an active result is indicative of a compound that increases activity in a target. Generally, an inactive result is indicative of a compound that decreases activity in a target.

In this example, data engine 111 uses experimental results 104 in initialing experimental space 118. Data engine 111 initializes experimental space 118 by annotating one or more of experiments 126 with observations, e.g., information indicative of an active result and/or with information indicative of an inactive result. In this example, an experiment is annotated with a solid, black circle for an active result. In this example, an experiment is annotated with a dashed line for an inactive result.

In this example, compound 122d has an inactive result on target 124a, e.g., as indicated by the dashed line for the experiment associated with compound 122d and target 124a. As shown in FIG. 1, compound 122b has an active result on target 124d. Compound 122d has an active result on target 124b. Compound 122e has an active result on target 124c. Compound 122g has an active result on target 124d.

In another example, data engine 111 may generate experimental results 104. In this example, data engine 111 generates experimental results 104 by randomly selecting a subset of targets 124a . . . 124n and a subset of compounds 122a . . . 122n. Data engine 111 executes experiments for each combination of target and compound that may be generated from the subsets. In this example, data engine 111 executes experiments by applying a compound to a target in a microtiter plate and measuring the results, including, e.g., measuring absorbance, fluorescence or luminescence as a reflection of target activity. Using observations (e.g., the results of the experiments), data engine 111 annotates one or more of experiments 126 with data indicative of the results, including, e.g., a dashed line and/or a solid, black circle.

Following initialization of experimental space 118, data engine 111 generates a model to represent available data in experimental space 1118. Using the model, data engine 111 selects additional experiments (e.g., additional compound-target pairs) to increase an accuracy of the model, e.g., relative to an accuracy of the model prior to execution of the additional experiments. Data engine 111 executes the additional experiments.

Data engine 111 collects data resulting from execution of the additional experiments. Using the collected data, data engine 111 updates experimental space 118 with data indicative of an observed outcome of an experiment. As previously described, data engine 111 annotates one or more of experiments 126 based on whether a compound increases or decreases activity in a target.

In an example, data engine 111 continues the above-described actions until the model achieves a desired level of accuracy, until a specified budget has been exhausted, until all experiments 126 have been annotated, and so forth. Generally, a budget refers to an amount of resources, including, e.g., computing power, bandwidth, time, and so forth.

In an example, the model generated by data engine 111 includes an active learning model. Generally, an active learning model includes a machine learning model that interactively queries an information source to obtain desired outputs at new data points.

In this example, data engine 111 is configured to generate various types of models, e.g., models that are independent of features of compounds 122a . . . 122n and targets 124a . . . 124n, models that are dependent on features of compounds 122a . . . 122n and targets 124a . . . 124n, and so forth. Generally, a feature includes a characteristic of an item, including, a characteristic of a target and/or of a compound.

Models that are Independent of Target and Compound Features

In an example, data engine 111 is configured to generate a model using experimental space 118 as initialized and results of additional experiments that are performed following initialization of experimental space 118. In this example, the model includes a predictive model that generates predictions of effects of compounds on targets. Using predictions of the model, data engine 111 is further configured to select a batch of experiments to further increase an accuracy of the model, e.g., relative to an accuracy of the model prior to performance of the batch of experiments.

Generation of Model that is Independent of Features

In an example, data engine 111 is configured to generate a model to predict an effect of compounds 122a . . . 122n on targets 124a . . . 124n. In this example, the model includes information defining a relationship between compounds 122a . . . 122n and targets 124a . . . 124n. In this example, data engine 111 generates the model by generating clusters of compounds 122a . . . 122n and targets 124a . . . 124n.

Data engine 111 executes a clustering technique to group together compounds 122a . . . 122n and targets 124a . . . 124n into one or more clusters. In this example, data engine 111 generates the clusters based on results of initialization of experimental space 118. For example, compound-target pairs associated with an inactive result may be grouped into one cluster. Compound-target pairs associated with an active result may be grouped into another cluster. From the clusters, data engine 111 generates the model by learning associations between compounds and targets in the various clusters.

In an example, data engine 111 implements an exploratory phase, in which data engine 111 learns information about each of compounds 122a . . . 122n and targets 124a . . . 124n. In this example, data engine 111 may implement experiments that include compounds 122a . . . 122n and/or targets 124a . . . 124n for which no information is known. For example, the information learned may include phenotypes. Generally, a phenotype includes observable physical and/or biochemical characteristics of an organism. In this example, data engine 111 generates clusters of compounds 122a . . . 122n and targets 124a . . . 124n, e.g., based on phenotypes of the compounds 122a . . . 122n and targets 124a . . . 124n.

In an example, data engine 111 may determine how a particular compound (e.g., compound 122a) perturbs various targets 124a . . . 124n. Targets 124a . . . 124n that are perturbed in similar ways may be related. Based on results of the perturbance, data engine 111 identifies phenotypes for targets 124a . . . 124n. In this example, the phenotypes include information indicative of a response by targets 124a . . . 124n to a perturbance caused by compound 122a. Using the phenotypes for targets 124a . . . 124n, data engine 111 generates clusters of targets 124a . . . 124n with similar phenotypes.

Using the clusters, data engine 111 generates a predictive model. For example, the predictive model may include a linear regression model. The linear regression model may be trained in accordance with the equations shown in the below Table 1:

TABLE 1

Y_{obs(*,p)P} = X_{obs(*,p)}βₚ^{P}
Y_{obs(d,*)D} = X_{obs(d,*)}β_{d}^{D}

As shown in the above Table 1, Y_{obs(*,p)} and X_{obs(*,p)} include matrices of measured activity levels and phenotypes respectively from all executed experiments with target p. Y_{obs(d,*)} and X_{obs(d,*)} include matrices of activity scores or phenotypes respectively from all executed experiments with compound d.

Data engine 111 selects a set of phenotypes that gives a fit where |β|<s. A penalty s is selected using cross validation for a linear regression model. Once a model has been trained, data engine 111 generates predictions for experiments using the equations shown in the below Table 2.

TABLE 2

Y_{(d,p)P} = Xₚβ_{d}^{D}
Y_{(d,p)D} = X_{d}β_{d}^{D}

In an example, data engine 111 generates a prediction for Y_{(d,p)} by taking the mean of the predictions shown in the above Table 2. A formula for generating a mean of the predictions is shown in the below Table 3:

TABLE 3

Y_{(d,p)P} = mean[Y_{(d,p)P} Y_{(d,p)D}]

As shown in the above Table 3, Y_{(d,p)P} includes a prediction of an effect of a compound on a target. In an example, the prediction includes an activity score. Generally, an activity score includes information indicative of a magnitude of an effect of a compound on a target. In this example, activity scores range from values of −100 to 100. A value of −100 is indicative of an inhabitation effect. In this example, an inhibition effect includes a type of inactive effect. A value of 100 is indicative of an activation effect, e.g., a compound that increases an activity level of a target. A value of zero is indicative of a neutral effect of the compound on the target.

In this example, experimental results 104 include activity scores. In this example, experimental space 118 is initialized with the activity scores included in experimental results 104, e.g., by populating one or more of experiments 126 with the activity scores. For example, experimental results 104 include information indicative of an activity score of compound 122d on target 124a. In this example, data engine 111 executes the model to generate activity scores for compound-target pairs that were not associated with results included in experimental results 104.

Batch Selection for Models that are Independent of Features

Using the model, data engine 111 selects additional experiments for execution (e.g., compound-target pairs for which there is no observed result). Data engine 111 implements various techniques in selecting the compound-target pairs.

In an example, data engine 111 uses predictions (e.g., activity scores or phenotype vectors) that were generated by the model in selecting a batch of experiments. In this example, data engine 111 executes a greedy algorithm that selects unexecuted experiments that have the greatest predicted effect (e.g., inhibition or activation) for measurement in an execution of the model. Generally, a greedy algorithm includes an algorithm that follows a problem solving heuristic of making a locally optimal choice at various stages of execution of the algorithm.

In another example, data engine 111 implements a clustering algorithm in selecting experiments. In this example, data engine 111 selects clusters of experiments, e.g., based on the predictions associated with the experiments. For a cluster, data engine 111 may be configured to select a predefined number of experiments that are located with increased proximity to a center of a cluster, e.g., relative to proximity of other experiments in the cluster.

Models that are Dependent on Target and Compound Features

In another example, data engine 111 retrieves, from data repository 105, information indicative of structures of targets 124a . . . 124n, including, e.g., an amino acid sequence. Using the structures, data engine 111 calculates features of targets 124a . . . 124n, including, e.g., molecular weight, theoretical isoelectric point, amino acid composition, atomic composition, extinction coefficient, instability index, aliphatic index, grand average of hydropathicity, and so forth.

In another example, data engine 111 retrieves additional features of targets 124a . . . 124n from data repository 105 and/or from another system (e.g., a system configured to run Protein Recon software). These features include estimates for density-based electronic properties of targets 124a . . . 124n, which are generated from a pre-computed library of fragments. In still another example, data engine 111 retrieves, from data repository 105, features indicating a presence or an absence of motifs in targets 124a . . . 124. In still another example, data engine 111 calculates features for compounds 122a . . . 122n, including, e.g., fingerprints. Generally, fingerprints include information indicative of a presence or an absence of a specific structural pattern.

In an example, the effects of features are additive in nature. In this example, data engine 111 is configured to generate a linear regression model, e.g., based on experimental space 118. In an example, each compound-target pair has associated with it a unique set of features. In this example, to generate a prediction for a compound-target pair, data engine 111 generates two independent predictions by training separate models (e.g., a linear regression model) for the compound and for the target. The model for a target is trained using the features and activity scores for all compounds which were observed with that target. The model for a compound is trained to predict which targets the compound would affect using the target features.

In this example, data engine 111 generates and trains a model in accordance with the formulas shown in the above Tables 1-3. In this example Y_{obs(*,p)P} and X_{obs(*,p)} include the matrices of activity scores and compound features respectively from all executed experiments with target p. Additionally, Y_{obs(d,*)D} and X_{obs(d,*)} include matrices of activity scores and target features respectively from all executed experiments with compound d.

Batch Selection for Models that are Dependent on Features

As previously described, data engine 111 uses the predictions in selecting experiments for execution, e.g., in another implementation of the model. Data engine 111 is configured to use numerous techniques in selecting experiments, including, e.g., a greedy algorithm, a density-based algorithm, an uncertainty sampling selection algorithm, a diversity selection algorithm, a hybrid selection algorithm, and so forth, each of which are described in further detail below.

In an example, data engine 111 implements a greedy algorithm in selecting experiments. In this example, data engine 111 selects experiments having a greatest absolute value of predicted activity score. In some examples, no information is available to make a prediction for an experiment. If no prediction is made from available data for an experiment, the experiment is predicted to have an activity score of zero. In this example, all experiments with equivalent activity scores are treated in random order.

In another example, data engine 111 implements a density-based selection algorithm. In this example, an experiment is represented by a single vector formed by concatenating the target features and the compound features for that experiment. In an example, to promote computational efficiency, a maximum of 2000 executed experiments and 2000 unexecuted experiments were used. Among the 2000 unexecuted experiments, data engine 111 makes selections using a density-based sampling method.

In still another example, data engine 111 implements an uncertainty sampling selection algorithm. For an unexecuted experiment, data engine 111 generates predictions using 5-fold cross validation for each model. In this example, data engine 111 calculates twenty-five predictions for each experiment, e.g., by calculating the mean of each compound prediction with each compound prediction. If calculation of a model is not possible, e.g., because of a lack of common observations, five predictions are used. Experiments are selected having the largest standard deviation of predictions.

In yet another example, data engine 111 implements a diversity selection algorithm. In this example, an experiment is represented by a single vector formed by concatenating the target features and the compound features for that experiment. A random set of experiments (e.g., 4000 experiments) are clustered using the k means algorithm (with k being the size of the batch desired). The experiment nearest to a centroid of a cluster is selected for execution.

In still another example, data engine 111 implements a hybrid selection algorithm. In a hybrid selection algorithm, data engine 111 selects a specified fraction of the experiments using each of the above-described methods.

### Detection of Hits

In another example, data engine 111 is configured to detect hits in experimental space 118. Generally, a hit includes an occurrence of a pre-defined event. In this example, each of compounds 122a . . . 122n and targets 124a . . . 124n are associated with a vector of features. In this example, a hit may include a compound that is associated with particular features and has a particular effect on a particular target (e.g., as indicated by an activity score). In this example, data engine 111 may be configured to use the model to generate predictions of effects of compounds on targets. Data engine 111 may then correlate the predictions with vectors of features for appropriate compounds and targets. Data engine 111 may compare the correlated predictions and features to various pre-defined events. Based on the comparison, data engine 111 may detect a hit, e.g., when the correlated predictions and features match one of the pre-defined events.

Batch Selection that is Independent of Models

In another example, data engine 111 is configured to select experiments independent of dynamic generation of a model. In this example, data engine 111 selects experiments based on features of compounds 122a . . . 122n and targets 124a . . . 124n.

In this example, data engine 111 retrieves information indicative of criteria for various batches of experiments. The criteria may be uploaded to data engine 111, e.g., by an administrator of network environment 100. In another example, data engine 111 may access the criteria from another system, e.g., a system that is external to network environment 100.

The criteria may specify that a batch include an equal sampling of different types of compounds. In an example, data engine 111 uses the features of compounds 122a . . . 122n to group together compounds 122a . . . 122n with similar features. In this example, a portion of compounds 122a . . . 122n that are grouped together are determined to be of a particular type. In this example, the criteria may specify that each batch of experiments include a predefined number of experiments for each type of compound. For example, if there are five different types of compounds. The criteria may specify that each batch include two experiments for each type of compound. In this example, the batch of experiments includes ten experiments.

In another example, data engine 111 selects experiments based on execution a sampling technique. In this example, the sampling technique is based on approximations to a hypergraph. Generally, a hypergraph includes a generalization of a graph, where an edge can connect any number of vertices. In an example, a hypergraph H includes a pair H=(X,E), where X is a set of elements, called nodes or vertices, and E is a set of non-empty subsets of X, called hyperedges or links. In this example, E includes a subset of [symbol] (X)\{∅}, where [symbol] (X) is the power set of X.

In still another example, the sampling technique includes an infima of the above-described active learning techniques. Generally, an infima includes a partially ordered set T (of a subset S) in which the greatest element of T is less than or equal to all elements of S. In this example, the sampling technique increases discoveries of experiments, while decreasing an amount of resources consumed in discovering the experiment.

In an example, the sampling technique uses statistical hypothesis testing guarantees, including, e.g., stopping rules. Generally, a stopping rule includes a mechanism for deciding whether to continue or stop a process on the basis of present position and past events.

In an example, the sampling technique determines a distribution (e.g., a discrete probability distribution) of probabilities of an experiment producing an effect (e.g., an active effect and/or an inactive effect). From the distribution, data engine 111 selects a predefined number of experiments associated with an increased probability of having an effect on a target, e.g., relative to other probabilities of other experiments.

In this example, the distribution includes a Poisson distribution. Generally, a Poisson distribution includes a distribution that expresses the probability of a given number of events occurring in a fixed interval of time and/or space if these events occur with a known average rate and independently of the time since the last event.

In another example, data engine 111 generates a distribution of experiments, e.g., based on the features of compounds 122a . . . 122n and targets 124a . . . 124n. In this example, data engine 111 selects experiments from the distribution to promote a balanced distribution of various types of experiments. In this example, the distribution includes various groups of experiments, e.g., experiments are grouped together based on the features of compounds 122a . . . 122n and targets 124a . . . 124n. In this example, data engine 111 is configured to select from each group a predefined number of experiments.

In yet another example, data engine 111 selects experiments using the following techniques. In an example, data engine 111 selects experiments for a set of compounds C and targets T. In this example, experimental space 118 includes observations of combinations (t, c)∈T×C. The set of sample paths over the experimental space 118 is the permutation group S|T×C|. An effective sampling strategy includes a computable function f such that for a uniformly convergent sequence of functions fₙ→f, in accordance with the equation in the below Table 4.

[ [symbol] _{ƒ}_{ₙ}[σ[k+b]σ[1 . . . k]]− [symbol] _{ƒ}[σ[k+1]][σ[1 . . . k]]]<ϵ Table 4

In an example, b is indicative of a batch of experiments. Given a maximum number of treatments that can be afforded (K<<|T×C|), data engine 111 is configured to sample from experimental space 118 so as to increase the quality of a sensible predictor constructed from the data.

In an example, experimental space 118 includes a natural geometry of a feature space induced over C, T. In an example, one or more of the above-described feature are used to describe variation in C. In this example, T includes one or more of the above described features.

In an example, data engine 111 is configured to discretize each feature Fᵢ for C (T) by some uniform means, for example Freedman-Diaconis' choice, producing bins F_{i,j}. Data engine 111 is further configured to associate, for each bin F_{i,j}, a c(t) with a Fᵢₜₕ feature in the bin. This discretization produces a finite (hypergraph) set system (V, S), with V=C×T and a Sⱼ□V□S for each bin F_{i,j} under the projection through c or t. In accordance with finite set systems: for each k≤K, a set A (|A|=k) is an ϵ-approximation for (V, S) for Sⱼ□S, in accordance with the formula shown in the below Table 5.

TABLE 5
$\vert \frac{\vert {S}_{j} \vert}{\vert V \vert} - \frac{\vert A ⋂ {S}_{j} \vert}{\vert A \vert} \vert \leq ϵ$

For the least ϵ, an ϵ-approximation A includes an even sample for each Sⱼ in the sense of proportional sampling, in accordance with the formula shown in the below Table 6.

TABLE 6

| [symbol] [(t,c) ϵ Sⱼ] − [symbol] [(t,c) ϵ Sⱼ[(t,c) ϵ A]| ≤ ε

Up to a constant factor, the size of any level set intersection may be estimated. Further, for each ϵ, there is an ϵ-approximation A of size O(ϵ⁻² log|S|)[4]. With an assumption about the statistics of the rank level sets (e.g., Poisson distributed), this produces a hypothesis test by the delta method.

In an example, data engine 111 constructs (V, S) using the above-described techniques. With a fixed batch size B to evenly divide |V|, data engine 111 constructs the following ϵ-approximations Aₙ for n□{0 . . . K} (e.g., K=|V|/B), as shown in the below Table 7.

TABLE 7
$\begin{matrix}{A}_{K} = V \\ {A}_{n} = {argmin }_{{A}_{n}} {min}_{ϵ} \vert \frac{\vert {S}_{j} \vert}{\vert V \vert} - \frac{\vert {A}_{n} ⋂ {S}_{j} \vert}{\vert {A}_{n} \vert} \vert - ϵ \\ {A}_{n} ⋐ {A}_{n + 1} \\ \vert {A}_{n} \vert = \vert {A}_{n + 1} \vert - B\end{matrix}$

As shown in the above Table 7, the sequence (Aₙ)ₙ□Σ describes a sample path that (i) is bounded variation for latent rank level sets away from the expected value over all Σ, and (ii) is data-dependent. Further, with smooth F_{i,j} intersections and a regression function, the sample path chosen simultaneously implements density and uncertainty sampling strategies without needing to compute a function over the ranks observed in sample course.

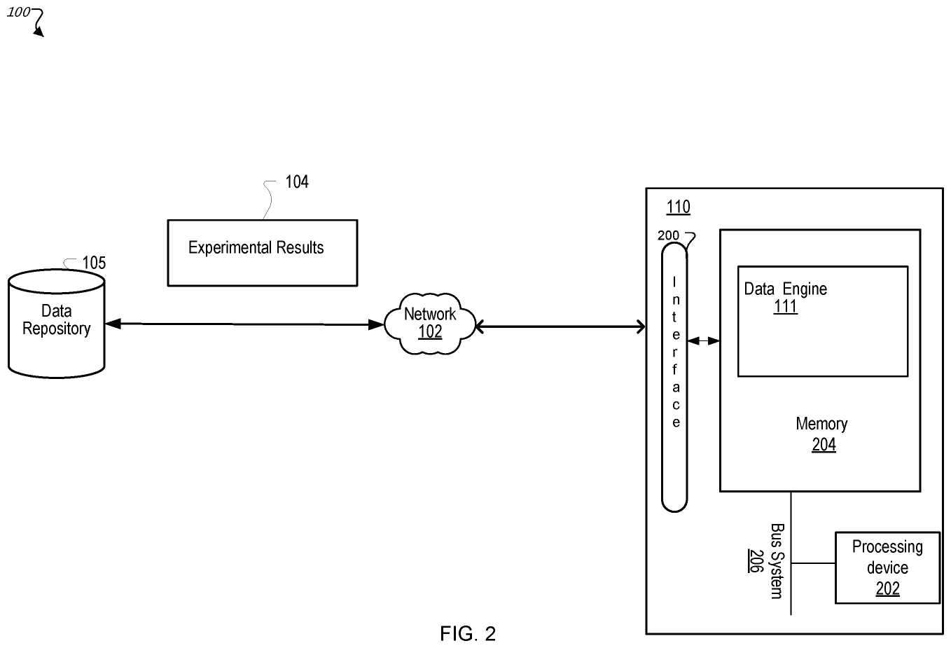

FIG. 2 is a block diagram showing examples of components of network environment 100 for generating predictions of effects of compounds 122a . . . 122n on targets 124a . . . 124n. In the example of FIG. 2, experimental space 118 is not shown.

Network 102 can include a large computer network, including, e.g., a local area network (LAN), wide area network (WAN), the Internet, a cellular network, or a combination thereof connecting a number of mobile computing devices, fixed computing devices, and server systems. The network(s) may provide for communications under various modes or protocols, including, e.g., Transmission Control Protocol/Internet Protocol (TCP/IP), Global System for Mobile communication (GSM) voice calls, Short Message Service (SMS), Enhanced Messaging Service (EMS), or Multimedia Messaging Service (MMS) messaging, Code Division Multiple Access (CDMA), Time Division Multiple Access (TDMA), Personal Digital Cellular (PDC), Wideband Code Division Multiple Access (WCDMA), CDMA2000, or General Packet Radio System (GPRS), among others. Communication may occur through a radio-frequency transceiver. In addition, short-range communication may occur, including, e.g., using a Bluetooth, WiFi, or other such transceiver.

Server 110 can be a variety of computing devices capable of receiving data and running one or more services, which can be accessed by data repository 105. In an example, server 110 can include a server, a distributed computing system, a desktop computer, a laptop, a cell phone, a rack-mounted server, and the like. Server 110 can be a single server or a group of servers that are at a same location or at different locations. Data repository 105 and server 110 can run programs having a client-server relationship to each other. Although distinct modules are shown in the figures, in some examples, client and server programs can run on the same device.

Server 110 can receive data from data repository 105 through input/output (I/O) interface 200. I/O interface 200 can be a type of interface capable of receiving data over a network, including, e.g., an Ethernet interface, a wireless networking interface, a fiber-optic networking interface, a modem, and the like. Server 110 also includes a processing device 202 and memory 204. A bus system 206, including, for example, a data bus and a motherboard, can be used to establish and to control data communication between the components of server 110.

Processing device 202 can include one or more microprocessors. Generally, processing device 202 can include an appropriate processor and/or logic that is capable of receiving and storing data, and of communicating over a network (not shown). Memory 204 can include a hard drive and a random access memory storage device, including, e.g., a dynamic random access memory, or other types of non-transitory machine-readable storage devices. As shown in FIG. 2, memory 204 stores computer programs that are executable by processing device 202. These computer programs include data engine 111. Data engine 111 can be implemented in software running on a computer device (e.g., server 110), hardware or a combination of software and hardware.

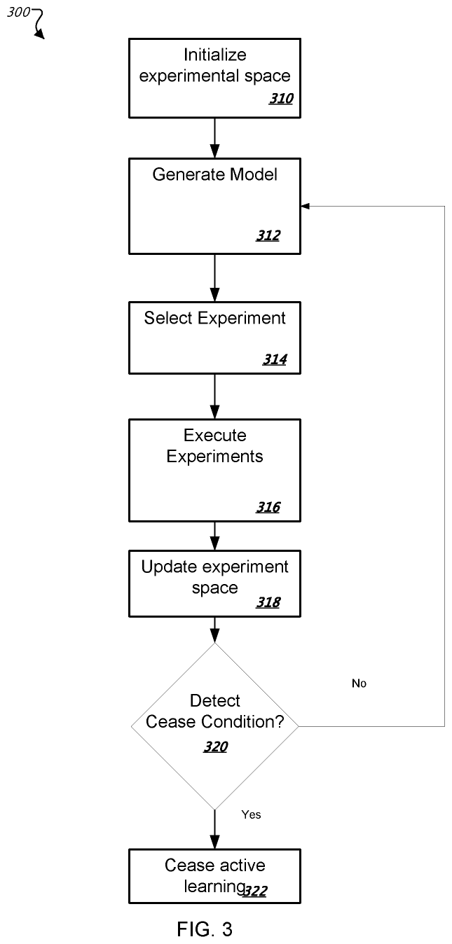

FIG. 3 is a flowchart showing an example process 300 for generating predictions of effects of compounds 122a . . . 122n on targets 124a . . . 124n. In FIG. 3, process 300 is performed on server 110 (and/or by data engine 111 on server 110).

In operation, data engine 111 initializes (310) experimental space 118. In an example, data engine 111 initializes experimental space 118 using experimental results 104. In this example, data engine 111 initializes experimental space 118 by determining a subset of experiments 126 for which experimental results 104 include observations. For the determined subset, data engine 111 annotates an experiment with the observation, e.g., information specifying whether a compound has an active or an inactive effect on a target. As described above, for an inactive effect, data engine 111 annotates an experiment with a dashed line. For an active effect, data engine 111 annotates an experiment with a solid, black circle.

In another example, data engine 111 initializes experimental space 118 by populating one or more of experiments 126 with activity scores (not shown in FIG. 1). In this example, experimental results 104 include activity scores for experiments performed on various compound-target pairs, including, e.g., a pair including compound 122b and target 124d.

In still another example, data engine 111 initializes experimental space 118 by annotating one or more of experiments 126 and by also populating the one or more of experiments 126 with activity scores included in experimental results 104. In this example, data engine 111 accesses threshold values for activity scores. For example, a threshold value may be zero. In this example, an activity score that exceeds the threshold value is indicative of an active effect. An activity score that is less than the threshold value is indicative of an inactive effect.

In the example of FIG. 3, data engine 111 generates (312) a model to predict effects of compounds on targets. In this example, the model generates predictions for unexecuted experiments, including, e.g., compound-target pairs for which an experiment has not been performed. For example, the model may generate predicted activity scores for unexecuted experiments.

As described above, data engine 111 may be configured to generate a model that is independent of features of compounds 122a . . . 122n and/or of targets 124a . . . 124n, e.g., as shown in the above Table 2. In another example, data engine 111 may be configured to generate a model that is based on features of compounds 122a . . . 122n and/or of targets 124a . . . 124n.

Data engine 111 selects (314) one or more unexecuted experiments for execution, e.g., based on the model. For example, data engine 111 may be configured to use predicted activity scores generated by the model in selecting experiments, e.g., based on an application of the greedy algorithm or one of the other above-described techniques. For example, data engine 111 may use the model in selecting experiments for the following compound-target pairs: compound 122b and target 124b, compound 122d and target 124f, compound 122i and target 124e, and so forth.

Data engine 111 executes (316) the selected experiments. During execution of the selected experiments, data engine 111 measures an effect of compounds on targets, e.g., the compounds and targets included in the experiments. In this example, data engine 111 measures an activity score for a compound-target pair by performing an experiment. The results of the experiment are converted to an activity, e.g., by converting a measured quantity to a percentage of a control condition. In another example, the results of an experiment may be converted to a phenotype vector containing the fractions of each of multiple patterns or components that are present in an image.

Data engine 111 updates (318) experimental space 118 with results (e.g., activity scores or phenotype vectors) of execution of the experiments. In an example, data engine 111 updates experimental space 118 by populating one or more of experiments 126 with results that were measured during the experiments. In this example, the update to experimental space 118 is used improve an accuracy of the model, e.g., by updating the model in accordance with the results of execution of the experiments.

Data engine 111 detects (320) whether a cease condition has been satisfied. Generally, a cease condition includes information indicative of a situation in which active learning is ceased. As previously described, data engine 111 may be configured to detect an occurrence of numerous cease conditions, including, e.g., a condition indicative of the model having achieved a desired level of accuracy, a condition indicative of a specified budget having been exhausted, a condition indicative of experimental space 118 including no more unexecuted experiments (e.g., all experiments in experimental space 118 have been performed), and so forth.

In an example, data engine 111 detects an absence of a cease condition. In this example, data engine 111 periodically repeats actions 312, 314, 316, 318, e.g., until data engine 111 detects a presence of a cease condition. In this example, an active learning technique includes a combination of actions 312, 314, 316, 318. In another example, data engine 111 detects a presence of a cease condition. In this example, data engine 111 is configured to cease (322) implementation of the active learning technique.

In a variation of FIG. 3, data engine 111 implements the techniques described above for batch selection that is independent of models. In this example, rather than selecting experiments based on predictions for unexecuted experiments, data engine 111 selects experiments based on features of compounds 122a . . . 122n and targets 124a . . . 124n. In this example, experiments may be selected prior to generation of the model.

Using the techniques described herein, a system generates predictions of effects of compounds on targets. The system generates a model for the predictions. The system implements numerous techniques in generating the model, including, e.g., techniques that generate the model independent of features of compounds 122a . . . 122n and targets 124a . . . 124n, techniques that generate the model based on features of compounds 122a . . . 122n and targets 124a . . . 124n, and so forth. Additionally, the system selects experiments to increase an accuracy of the model, based on predictions generated by the model.

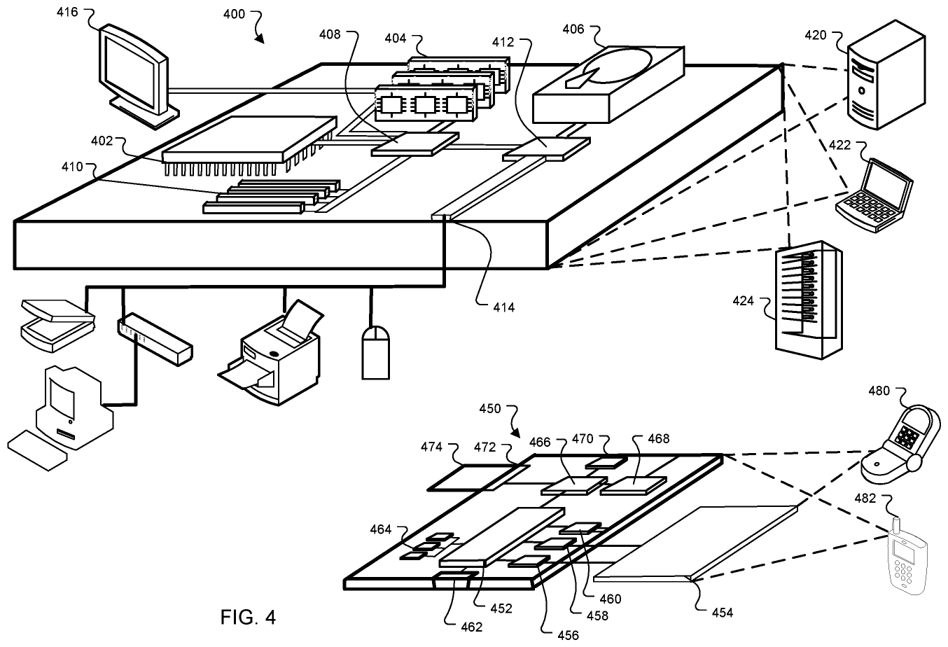

FIG. 4 shows an example of computer device 400 and mobile computer device 450, which can be used with the techniques described here. Computing device 400 is intended to represent various forms of digital computers, such as laptops, desktops, workstations, personal digital assistants, servers, blade servers, mainframes, and other appropriate computers. Computing device 450 is intended to represent various forms of mobile devices, such as personal digital assistants, cellular telephones, smartphones, and other similar computing devices. The components shown here, their connections and relationships, and their functions, are meant to be examples only, and are not meant to limit implementations of the techniques described and/or claimed in this document.

Computing device 400 includes processor 402, memory 404, storage device 406, high-speed interface 408 connecting to memory 404 and high-speed expansion ports 410, and low speed interface 412 connecting to low speed bus 414 and storage device 406. Each of components 402, 404, 406, 408, 410, and 412, are interconnected using various busses, and can be mounted on a common motherboard or in other manners as appropriate. Processor 402 can process instructions for execution within computing device 400, including instructions stored in memory 404 or on storage device 406 to display graphical data for a GUI on an external input/output device, such as display 416 coupled to high speed interface 408. In other implementations, multiple processors and/or multiple buses can be used, as appropriate, along with multiple memories and types of memory. Also, multiple computing devices 400 can be connected, with each device providing portions of the necessary operations (e.g., as a server bank, a group of blade servers, or a multi-processor system).

Memory 404 stores data within computing device 400. In one implementation, memory 404 is a volatile memory unit or units. In another implementation, memory 404 is a non-volatile memory unit or units. Memory 404 also can be another form of computer-readable medium, such as a magnetic or optical disk.

Storage device 406 is capable of providing mass storage for computing device 400. In one implementation, storage device 406 can be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in a data carrier. The computer program product also can contain instructions that, when executed, perform one or more methods, such as those described above. The data carrier is a computer- or machine-readable medium, such as memory 404, storage device 406, memory on processor 402, and the like.

High-speed controller 408 manages bandwidth-intensive operations for computing device 400, while low speed controller 412 manages lower bandwidth-intensive operations. Such allocation of functions is an example only. In one implementation, high-speed controller 408 is coupled to memory 404, display 416 (e.g., through a graphics processor or accelerator), and to high-speed expansion ports 410, which can accept various expansion cards (not shown). In the implementation, low-speed controller 412 is coupled to storage device 406 and low-speed expansion port 414. The low-speed expansion port, which can include various communication ports (e.g., USB, Bluetooth®, Ethernet, wireless Ethernet), can be coupled to one or more input/output devices, such as a keyboard, a pointing device, a scanner, or a networking device such as a switch or router, e.g., through a network adapter.

Computing device 400 can be implemented in a number of different forms, as shown in the figure. For example, it can be implemented as standard server 420, or multiple times in a group of such servers. It also can be implemented as part of rack server system 424. In addition or as an alternative, it can be implemented in a personal computer such as laptop computer 422. In some examples, components from computing device 400 can be combined with other components in a mobile device (not shown), such as device 450. Each of such devices can contain one or more of computing device 400, 450, and an entire system can be made up of multiple computing devices 400, 450 communicating with each other.

Computing device 450 includes processor 452, memory 464, an input/output device such as display 454, communication interface 466, and transceiver 468, among other components. Device 450 also can be provided with a storage device, such as a microdrive or other device, to provide additional storage. Each of components 450, 452, 464, 454, 466, and 468, are interconnected using various buses, and several of the components can be mounted on a common motherboard or in other manners as appropriate.

Processor 452 can execute instructions within computing device 450, including instructions stored in memory 464. The processor can be implemented as a chipset of chips that include separate and multiple analog and digital processors. The processor can provide, for example, for coordination of the other components of device 450, such as control of user interfaces, applications run by device 450, and wireless communication by device 450.

Processor 452 can communicate with a user through control interface 458 and display interface 456 coupled to display 454. Display 454 can be, for example, a TFT LCD (Thin-Film-Transistor Liquid Crystal Display) or an OLED (Organic Light Emitting Diode) display, or other appropriate display technology. Display interface 456 can comprise appropriate circuitry for driving display 454 to present graphical and other data to a user. Control interface 458 can receive commands from a user and convert them for submission to processor 452. In addition, external interface 462 can communicate with processor 442, so as to enable near area communication of device 450 with other devices. External interface 462 can provide, for example, for wired communication in some implementations, or for wireless communication in other implementations, and multiple interfaces also can be used.

Memory 464 stores data within computing device 450. Memory 464 can be implemented as one or more of a computer-readable medium or media, a volatile memory unit or units, or a non-volatile memory unit or units. Expansion memory 474 also can be provided and connected to device 450 through expansion interface 472, which can include, for example, a SIMM (Single In Line Memory Module) card interface. Such expansion memory 474 can provide extra storage space for device 450, or also can store applications or other data for device 450. Specifically, expansion memory 474 can include instructions to carry out or supplement the processes described above, and can include secure data also. Thus, for example, expansion memory 474 can be provide as a security module for device 450, and can be programmed with instructions that permit secure use of device 450. In addition, secure applications can be provided via the SIMM cards, along with additional data, such as placing identifying data on the SIMM card in a non-hackable manner.

The memory can include, for example, flash memory and/or NVRAM memory, as discussed below. In one implementation, a computer program product is tangibly embodied in a data carrier. The computer program product contains instructions that, when executed, perform one or more methods, such as those described above. The data carrier is a computer- or machine-readable medium, such as memory 464, expansion memory 474, and/or memory on processor 452, that can be received, for example, over transceiver 468 or external interface 462.

Device 450 can communicate wirelessly through communication interface 466, which can include digital signal processing circuitry where necessary. Communication interface 466 can provide for communications under various modes or protocols, such as GSM voice calls, SMS, EMS, or MMS messaging, CDMA, TDMA, PDC, WCDMA, CDMA2000, or GPRS, among others. Such communication can occur, for example, through radio-frequency transceiver 468. In addition, short-range communication can occur, such as using a Bluetooth®, WiFi, or other such transceiver (not shown). In addition, GPS (Global Positioning System) receiver module 470 can provide additional navigation- and location-related wireless data to device 450, which can be used as appropriate by applications running on device 450.

Device 450 also can communicate audibly using audio codec 460, which can receive spoken data from a user and convert it to usable digital data. Audio codec 460 can likewise generate audible sound for a user, such as through a speaker, e.g., in a handset of device 450. Such sound can include sound from voice telephone calls, can include recorded sound (e.g., voice messages, music files, and the like) and also can include sound generated by applications operating on device 450.

Computing device 450 can be implemented in a number of different forms, as shown in the figure. For example, it can be implemented as cellular telephone 480. It also can be implemented as part of smartphone 482, personal digital assistant, or other similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms machine-readable medium and computer-readable medium refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions.

To provide for interaction with a user, the systems and techniques described here can be implemented on a computer having a display device (e.g., a CRT (cathode ray tube) or LCD (liquid crystal display) monitor) for displaying data to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (LAN), a wide area network (WAN), and the Internet.

The computing system can include clients and servers. A client and server are generally remote from each other and typically interact through a communication network. The relationship of client and server arises by virtue of computer programs running on the respective computers and having a client-server relationship to each other.

In some implementations, the engines described herein can be separated, combined or incorporated into a single or combined engine. The engines depicted in the figures are not intended to limit the systems described here to the software architectures shown in the figures.

A number of embodiments have been described. Nevertheless, it will be understood that various modifications can be made without departing from the spirit and scope of the processes and techniques described herein. In addition, the logic flows depicted in the figures do not require the particular order shown, or sequential order, to achieve desirable results. In addition, other steps can be provided, or steps can be eliminated, from the described flows, and other components can be added to, or removed from, the described systems. Accordingly, other embodiments are within the scope of the following claims.

What is claimed is:

1. A method performed by one or more processing devices, comprising:
obtaining information indicative of experiments associated with combinations of targets and compounds;
initializing the information with a result of at least one of the experiments;
generating, based on initializing, a model to predict effects of the compounds on the targets;
generating, based on the model and the experiments obtained, predictions for experiments to be executed;
selecting, based on the predictions, one or more experiments from the experiments to be executed;
executing the one or more experiments; and
updating the model with one or more results of execution of the one or more experiments.
